(19) United States
(12) Patent Application Publication
(10) Pub. No.: US 2023/0189815 A1
(43) Pub. Date: Jun. 22, 2023

(54) Bacterial strain Bacillus mojavensis PS17 for producing higher yield and protection of agricultural plants against phytopathogenic fungi

(71) Applicant: Organic Park Limited Limied Liablility Company, Kazan (RU)

(72) Inventors: Radik Ilyasovich SAFIN, Kazan (RU); Liliya Zyadatovna KARIMOVA, Kazan (RU); Shami Zavdatovich VALIDOV, Kazan (RU); Emest KOMISSAROV, Kazan (RU); Roderick DIABANKANA, Naberezhnnye Chelny (RU)

(73) Assignee: Organic Park Limited Limied Liablility Company, Kazan (RU)

(21) Appl. No.: 17/781,930

(22) PCT Filed: Nov. 19, 2020
(86) PCT No.: PCT/RU2020/050333

§ 371 Date: Jun. 2, 2022

(30) Foreign Application Priority Data
Dec. 13, 2019 (RU) 2019141759

(51) Int. Cl. A01N 63/22; C12N 1/20; A01P 21/00; A01P 3/00

(52) U.S. Cl. CPC A01N 63/22; A01P 3/00; A01P 21/00; C12N 1/205; C12R 2001/07

(57) ABSTRACT

The proposed solution refers to agricultural biotechnology and microbiology. It represents Bacillus mojavensis PS17 strain that was isolated from the seeds of the Sadokat variety of spring wheat and was deposited in the National Bioresource Center - Russian National Collection of Industrial Microorganisms (NBC VKPM), NRC “Kurchatov Institute” - GosNIIgenetika under registration number VKPM B-13415. The strain has a broad spectrum of activity against plant pathogenic fungi and improves the yield of agricultural crops. The strain is non-toxic to animals, exhibits growth-stimulating activity for plants and can be used to produce of biopreparations on the basis thereof for the protection of plants from diseases caused by fungal pathogens.

### CROSS-REFERENCE TO RELATED APPLICATIONS

This application is a U.S. national stage application of an international application PCT/RU2020/50333 filed on 19 Nov. 2020, published as WO/2021/118412, which international application claims priority of a Russian Federation patent application RU2019141759 filed on 13 Dec. 2019.

### FIELD OF THE INVENTION

The invention lays in the field of agricultural microbiology and biotechnologies, particularly, in the field of production of biological preparations for protection of crops against plant pathogenic fungi, and can be used to create biological fungicides to promote growth and increase yield of agricultural crops and represents bacterial strain Bacillus mojavensis PS17.

### BACKGROUND OF THE INVENTION

Many plant pathogenic fungi cause substantial damage to yield and qualitative characteristics of agricultural crops [1]. In order to protect crops against diseases caused by fungi, chemical and biological preparations called fungicides [2] are used. Application of chemical fungicides involves a number of negative aspects connected with environmental problems and development of immunity (resistance) against them in phytopathogens [3]. In this connection, effective biological protection of crops has assumed importance [4]. To resolve the task, biological preparations containing various microorganisms are used most frequently. Bacteria of Bacillus and Pseudomonas genus [5] are used broadly as biological agents in biopreparations for plant protection against diseases, of which, may represent a portion of natural microbial flora of plants, thus interacting with them beneficially. Despite the fact that many microorganisms can promote plants growth and protect them against of phytopathogenic organisms, spore-forming bacteria of Bacillus genus have an advantage in terms of ease of application, since their spores can store for a long period, which permits biopreparations containing such microorganisms to keep their activity longer and effective industrial process for their production to be developed [6]. Positive effect of bacterial strains of Bacillus on growth and development of plants, decrease in disease development and increase in yield have been determined [7]. In this connection, close attention is paid to searching of effective bacterial strains of Bacillus.

Bacterial strains of genus Bacillus are known as possess ability to suppress growth of plant pathogenic fungi and promote formation of yield of agricultural crops. In particular, this refers to Bacillus subtilis VNIISKhM 128 and biological preparation Phytosporin produced on basis of the same strain [8]; B. subtilis B 93 VIZR [9], B. subtilis BZR 517 [10], etc.

Disadvantage of all of the above listed strains is insufficient biological activity in terms of suppression of phytopathogens and scarcely studied effect on yield of agricultural crops.

One valuable source to search for biological agents for creation of biopreparations for plant protection against deseases is endophytic bacteria [11].

Bacillus mojavensis Lhv-97 strain is the closest to the proposed invention, that possesses fungicidal and bactericidal activity and is isolated from rhizosphere soil [12].

### OBJECT AND BRIEF DESCRIPTION OF THE INVENTION

The purpose of the invention is to determine strain that can be used to attain biological preparation with high activity in terms of suppression of plant pathogenic fungi and increasing of yield of agricultural crops, enabling broadening of a set of means of biological protection of plants against diseases.

The purpose set is resolved through the fact that Bacillus mojavensis PS17 strain which can be used to attain biopreparation against plant pathogenic fungi and increase yield of agricultural crops.

The new strain was isolated from the seeds of the Sadokat variety of spring wheat (Republic of Tajikistan) and deposited in the National Bioresource Center -Russian National Collection of Industrial Microorganisms (BRC VKPM), NRC “Kurchatov Institute” -GosNIIgenetika, under registration number VKPM B-13415.

The principal selection criteria included suppression of growth of plant pathogenic fungi, positive effect on crop productivity, absence of pathogenicity to homeothermic animals and compatibility with other microorganisms. Species membership was determined with use of molecular genetic methods by sequence of nucleotides in 16S rNRA, as well as by amplification of species-specific fragment that is characteristics for bacteria of genus Bacillus mojavensis in NRC “Kurchatov Institute” - GosNIIgenetika.

The strain possesses the following specific morphological-cultural and physiological-biochemical features. Strain cells represent gram-positive aerobic spore-forming rod-shaped straight cells with rounded ends and dimensions 1.5-2.5×0.5-0.7 µm; they are positioned normally in couples or as a single unit; chains are encountered more rarely. Cells do not swell during spore formations; spores are ellipsoidal and are centrally located.

On meat infusion agar (MIA), after 2 days, spherical colonies with a scalloped border are formed, which are 5-6 mm in diameter; they mainly have grained surface, and are mat, obscure, the color is white with greenish shades. Colonies have convex bottom, relief foldings and substantially-raised central crater; they have viscous, gummy consistency; they have a soft outer coriaceous layer and do not grow into agar.

Bacillus mojavensis PS17 represent aerobic chemo-organotrophic bacterium which need no growth factors. It grows within the temperature range of 10 to 47° C. with optimal range 28-32° C., at pH from 4.5 to 8.5 with the optimum of 7.0-7.5, and at concentration of sodium chloride up to 5%.

It demonstrates activity of tryptophane desaminase and gelatinase. Activity of β-galactosidase (ortonitrophenyl-βD-galactopyramosidase), arginine dihydrolase, lysin decarboxylase, ornithin decarboxylase, is not determined. It does not produce indole and hydrogen sulphide, and reduces nitrates. Voges-Proskauer reaction (acetoin production) is positive.

As a source of carbon and energy, it utilizes D-glucose, D-fructose, D-xylose, D-ribose, L-arabinose, D-mannose, D-mannite, inositol, D-sorbitol, methyl-αD-mannopyranosyde, methyl-αD-glucopyranosyde, D-maltose, D-cellobiose, D-lactose, amygdalin, arbutine, aesculin, salicin, D-melibiose, D-sucrose, D-trehalose, D-raffinose, gentibiose, glycerin, citrate.

It does not utilize erythritol, D-arabinose, D-adonitol, D-galactose, L-xylose, L-sorbose, L-rhamnose, methyl-βD-xilopyranoside, dulcitol, N-acetylglucosamin, inulin, D-melezitose, xylitol, D-turanose, D-lyxose, D-tagatose, D-fucose, D-arabitol, L-arabitol, potassium gluconate, potassium 2-cetogluconate, potassium 5-cetogluconate.

The strain is stored at 4-6° C. in test tubes with semisolid agar in mineral medium (composition: dibasic potassium phosphate 5.8 g/L, monobasic potassium phosphate 3 g/L, ammonium sulphate 1 g/L, glycerin 2 g/L with addition of 0.7% agar, pH 7.0-7.2) under paraffinic oil, which is poured in test tubes upon expiration of the 2nd day of culture growth. Under the above conditions, storage life of strain without subculturing is at least 1 year.

Strain grows well on meat infusion agar (MIA), King B, LB, wort agar (composition: concentrate of unhopped beer wort diluted with distilled water to total sugar concentration of 10% (10° Balling), pH 7.0-7.2 with addition of 2.0% agar agar), Gramyko medium, Gause medium no.2 (composition: tripton 2.5 g; pepton 5.0 g; NaCI 5.0 g; glucose 10.0 g; agar agar 20 g; tap water 1000 mL; pH - 7.0-7.4) and mineral medium (composition: dibasic potassium phosphate 5.8 g/L, monobasic potassium phosphate 3 g/L, ammonium sulphate 1 g/L, glycerin 10 g/L with addition of 0.7% agar, medium pH 7.0-7.2). Fermentation is performed on mixture of equal volumes of meat pepton broth and 6° unhopped wort (pH 6.9-7.2) at 30° C. up to 95% of spore formation. Quantity of CFU is at least 5×10⁹ per 1 mL.

Study of pathogenicity of the claimed strain for homeotherm animals was conducted at GBOU VPO Kazan Federal University, which resulted in the conclusion that, by the indicators of virulence, dissemination, toxicity and toxigenicity, Bacillus mojavensis PS17 strain is not pathogenic for homeotherm animals and meets the requirements set for industrial microorganisms.

### EXEMPLARY EMBODIMENTS OF THE INVENTION

### Example 1 Study of Antagonistic Activity of Bacillus Mojavensis PS17 Strain

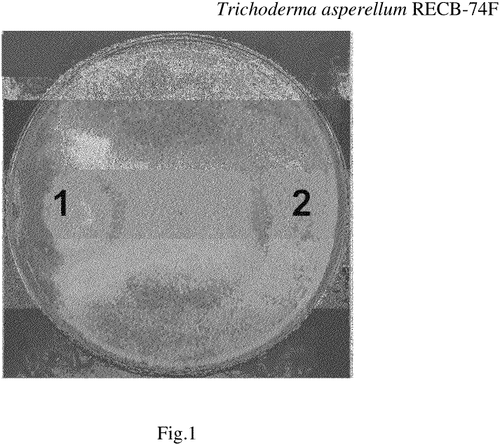

Antagonistic activity of Bacillus mojavensis PS17 strain was studied with use of confrontational method. The block with mycelium of tested fungus was put in the centre of Petri dishes with medium King B, and, by injection, strain Bacillus mojavensis PS17 was planted at a distance of 2-3 cm; as negative control, strain Pseudomonas putida PCL1760 was used, which was also planted by injection, and which does not produce any antagonistic substance suppressing development of micromycetes (FIGS. 1-5). Petri dishes were placed for 48-72 hours into thermostat under temperature 25° C. so that micromycetes and bacteria can develop. Observations were held on Day 2 to 5 depending on growth rate of phytopathohen.

The method of determination of antagonistic activity is considered as qualitative, where presence or absence of antagonistic properties is determined by occurrence of zones of growth suppression of test cultures around colonies of tested microorganisms. This is connected to the fact that in these conditions of test setup, antagonism between microorganisms is realized through production of various antimicrobial metabolites by them. In their turn, the metabolites infuse in feed medium, therefore their accumulation in a specific point of feed medium and, accordingly, a size of zone where manifestations of antagonism are observed, depends of a volume of medium, as well as on quality and density of agar.

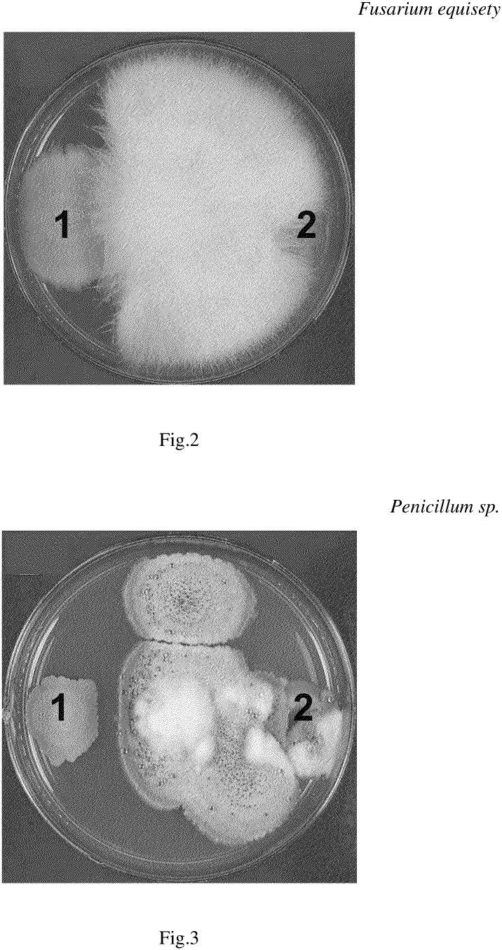

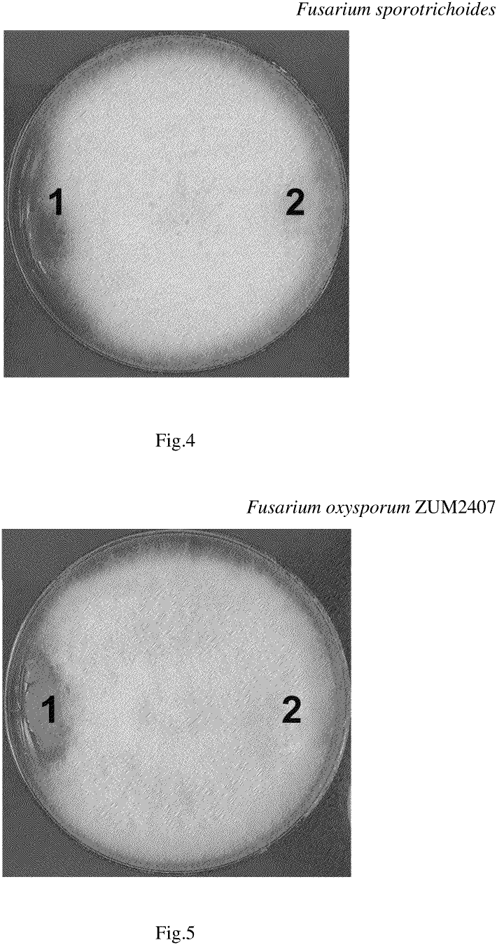

The results of determination of antagonistic activity of Bacillus mojavensis PS17 strain are summarized in FIGS. 1-5. There, the following are represented as variants: 1) - Bacillus mojavensis PS17 strain, 2) Pseudomonas putida PCL1760. As test culture of micromicetes, the following are used: FIG. 1 - Trichoderma asperellum RECB-74F, FIG. 2 - Fusarium equisety, FIG. 3- Penicillum sp., FIG. 4 - Fusarium sporotrichoides, FIG. 5- Fusarium oxysporum ZUM2407.

The results of determination of antagonistic activity demonstrated that all test cultures of micromycetes used in the study showed sensitivity to metabolites of Bacillus mojavensis PS 17 strain, which manifested as occurrence of zones of growth of test cultures. According to the information provided, Bacillus mojavensis PS17 strain is antagonist and possesses broad spectrum of activity.

### Example 2. Laboratory Tests for Phytotoxicity and Growth-Promoting Activity of Bacillus mojavensis PS17 strain.

For testing of phytotoxicity and growth-promoting properties of Bacillus mojavensis PS17 strain, were used wheat grains of grade Simbircite. To do so, grains were soaked in cell suspension grown on rich LB medium, which consists of 10 g/L of bactotrypton, 5 g/L of yeast extract and 10 g/L of sodium chloride. The ready medium was sterilized for 40 minutes under 110° C. After soaking, seeds have been placed in a vessel on filter paper wetted with tap water. Seeds have been incubated for 7 days under temperature of 23° C. with photoperiod of 16 hours. The plants were collected and the length of the roots was measured. The results of experiments demonstrated that Bacillus mojavensis PS17 strain added lengths of wheat stems and roots (Table 1).

TABLE 1

Effect of Bacillus mojavensis PS17 on biometrics of wheat crops of grade Simbircite

| Variant | Experiment 1 | Experiment 2 |
|---|---|---|
| Wheat grass | | |
| Control | 142.9±2.9 | 141.6±3.6 |
| Bacillus mojavensis PS17 | 146.5±14.9 | 151.6±6.3 |
| Wheat roots | | |
| Control | 100.9±5.8 | 102.5±2.4 |
| Bacillus mojavensis PS17 | 127.5±7.7 | 126.0±8.9 |

### Example 3 Compatibility With Beneficial Microorganisms

Study of compatibility of Bacillus mojavensis PS17 strain with beneficial bacteria Pseudomonas fluorescens RECB-14B, Streptomyces resistomycificus RECB-31B, Pseudomonas fluorescens RECB-44B, Bacillus mojavensis RECB-50B, Trichoderma asperellum RECB-74F on rich and minimal media. As a result of confrontation tests, it was shown that from the specified list of beneficial microorganisms, the Bacillus mojavensis PS 17 strain suppresses the growth of Trichoderma asperellum RECB-74B, Streptomyces resistomycificus RECB-31B and Bacillus amyloliquifaciens RECB-95B strains. At the same time, the growth of Bacillus mojavensis PS17 strain was suppressed by Streptomyces resistomycificus RECB-31B and Bacillus amyloliquifaciens RECB-95B strains. Absence of suppression of growth of strains Pseudomonas fluorescens RECB-14B and Pseudomonas fluorescens RECB-44B by Bacillus mojavensis PS17 strain and, vice versa, absence of suppression of growth of Bacillus mojavensis PS17 by Pseudomonas fluorescens RECB-14B and RECB-44B strains indicate possibility to use this microorganism with other pseudomonas-based biologics.

### Example 4. Evaluation of Efficiency of Treatment of Crops of Summer Wheat With Bacillus Mojavensis PS17 Strain In Field Conditions

The studies were conducted in experimental fields of the Kazan State Agrarian University. The subject of study was spring wheat grade Ecada 66. Efficiency of crop spraying during growing period was studied. As experimental variant, biopreparation on basis of Bacillus mojavensis PS17 was used with consumption rate of 1.0 L/ha. Production of the biopreparation was held on laboratory shaking machine Innova 40R (Eppendorf) under temperature of 37° C., incubation period 17 hours at rotation of 180 rpm. Medium to make preparations was liquid feed medium LB, Luria broth. Titer was at least 2.5*109 CFU/ml Variant threated with water was used as control. Standard was registered biological preparation Rhizoplane on basis of Pseudomonas fluorescens strain AP-33 with consumption rate of 1 L/ha. Agrotechnology of spring wheat growing was generally accepted for the zone of Predkamie in the Republic of Tatarstan (Russia). Consumption rate of tank mix was 200 L/ha. Treatment was conducted twice, during paniculation stage and blooming stage of wheat.

Use of different treatment options affected development of leaf mycotic diseases (Table 2) and prevalence of ears (Table 3) with diseases.

TABLE 2

Development of leaf diseases and biological efficiency of their control with use of bioagents on summer wheat (in 14 days after treatment), %, 2018

| Variant | Development of disease, % | | Biological efficiency, % | |
|---|---|---|---|---|
| | Septoria leaf blotch | true mildew | Septoria leaf blotch | true mildew |
| Control | 13.0 | 2.5 | | |
| Rhizoplane | 15.1 | 1.0 | 0 | 60.0 |
| Bacillus mojavensis PS17 | 10.0 | 0.5 | 23.1 | 80.0 |

In terms of control of leaf diseases, use of Bacillus mojavensis PS17 caused a more prominent effect on decreasing of development of Septoria leaf blotch and true mildew as compared to standard biofungicide Rhizoplane.

TABLE 3

Prevalence of ear diseases and biological efficiency of their control with use of bioagents on summer wheat during dough stage, %, 2018

| Variant | Development of disease, % | | Biological efficiency, %8 | |
|---|---|---|---|---|
| | dark mildew | Septoria leaf blotch | dark mildew | Septoria leaf blotch |
| Control | 2.0 | 5.1 | | |
| Rhizoplane | 1.5 | 2.1 | 25.0 | 58.8 |
| Bacillus mojavensis PS17 | 1.1 | 1.8 | 45.0 | 64.7 |

In terms of control of ear diseases, preparation on basis of Bacillus mojavensis PS17 surpassed significantly standard biofungicide Rhizoplane by efficiency of control of mycotic diseases.

Treat of crops with Bacillus mojavensis PS17 conclusively increased yield of spring wheat by 0.53 t/ha, which significantly exceeds the results obtained with use of standard biofungicide Rhizoplane (Table 4).

TABLE 4

Yield of summer wheat of grade Ecada 66, t/ha, 2018

| Variant | Yield, t/ha | Yield increment | |
|---|---|---|---|
| | | t/ha | % |
| Control | 2.48 | | |
| Rhizoplane | 2.57 | 0.09 | 3.8 |
| Bacillus mojavensis PS17 | 3.01 | 0.53 | 21.3 |
| HCP₀₅ | 0.08 | | |

Therefore, using double treatment during crop growing period of endophytic bacteria Bacillus mojavensis PS17 with consumption rate of 1 L/ha causes prominent effect on decrease of attack by main diseases on spring wheat crops and results in growth of yield.

### Example 5. Evaluation of Efficiency of Pre-plant Treatment of Potato Tubers With Bacillus Mojavensis PS 17 Strain In Field Conditions

Subject of study was potato grade Veneta, reproduction of super-elite seeds. Options of tuber treatment before planting were studied. Experiment layout: 1. Control - without treatment. 2. Chemical preparation standard - 290 sc (imidacloprid + pencycuron) Prestige (0.7 L/t). 3. Biological preparation - Rhizoplane on basis of Pseudomonas fluorescens AP-33 (1 L/t). 4. Biopreparation on basis of endophytic bacterium Bacillus mojavensis PS17 (1.0 L/t). Number of replications in experiments - four times. Consumption rate of working liquid - 20 L/t. Tubers were treated immediately before planting.

The results of treatment for prevalence of sprouts with brown patch is represented in Table 6.

TABLE 6

- Prevalence of potato sprouts with brown patch and biological efficiency of control with use of tuber treatment, %, 2018.

| Variant | Disease count | | Biological efficiency, % | |
|---|---|---|---|---|
| | P* | R** | P | R |
| Control | 27.4 | 6.4 | | |
| Prestige | 5.1 | 0.6 | 81.4 | 90.6 |
| Rhizoplane 12.1 2.4 55.8 62.5 | | | | |
| Bacillus mojavensis PS17 | 5.5 | 0.7 | 79.9 | 89.1 |

Note: * P - prevalence of disease, %; ** R - development of disease, %.

In terms of efficiency of control of brown patch, preparation in basis of Bacillus mojavensis PS17 surpassed standard biofungicide Rhizoplane and underperformed insignificantly as compared to chemical preparation Prestige.

Maximum productivity (Table 7) was obtained when preparation on basis of Bacillus mojavensis PS17 was used. While as compared to chemical standard, no plausible difference in terms of yield was found in the variant, then, as compared to standard biofungicide Rhizoplane, total yield increment made 3.1 t/ha.

TABLE 7

Yield of potato grade Veneta with use of tuber treatment, t/ha, 2018.

| Variant | Yield, t/ha | Addition to control | |
|---|---|---|---|
| | | t/ha | % |
| Control | 15.5 | | |
| Prestige | 23.3 | 7.8 | 50.2 |
| Rhizoplane | 20.6 | 5.1 | 33.0 |
| Bacillus mojavensis PS17 | 23.7 | 8.2 | 53.0 |
| HCP₀₅ | 0.08 | | |

Therefore, bacterial strain Bacillus mojavensis PS17 possesses fungicidal activity with respect to plant pathogenic fungi and increases yield of agricultural crops, which will permit to broaden the spectrum of means of biological protection of crops against diseases.

### LIST OF REFERENCES

1. Chumakov, A.E. Harmfulness of diseases of agricultural crops / A.E. Chumakov, T.I. Zakharova. - M.: Agropromizdat, 1990. - 127 pages.

2. Shkalikov, V.A. Crop protection against diseases: studybook/ V.A. Shkalikov, O. O. Beloshapkina, D. D. Bukreyev, etc.; Edited by V.A.Shkalikov. - M.: Koloss, 2010. - 404 pages.

3. Tyuterev, S.L. Modes of action of fungicides on plant pathogenic fungi/ S. L. Tyuterev; Russian Academy of Agricultural Sciences, All-Russian R&D Institute of Crop Protection. - Saint-Petersburg: Niva, 2010. - 170 pages.

4. Shternshis, M.V. Biological protection of plants: Studybook/M.V. Shternshis, I.V. Andreyeva, O. G. Tomilova. - SPb.: Lan Punlishers, 2019. - 332 pages.

5. Shternshis, M.V. Tendencies of development of biotechnology of bacterial means of plant protection in Russia/M.V. Shternshis// Vestnik of Tomsk State University. Biology. -2012. - Issue 2 (18). - P. 92-100.

6. Khomyak, A.I. Optimal growing conditions of new bacteria of genus Bacillus that are promising for development of biofungicides on their basis / A.I. Khomyak, A.M. Asaturova, T.M. Sidoriva// In compilation: Biological plant protection as the basis for agricultural ecosystem stabilization. Materials of reports presented to the 8th International Conference. -2014. - P. 298-301.

7. Asaturova A.M. Studies on effect of seed bacterization on growth and development of winter wheat crops / A.M. Asaturova, V.D. Nadykta, V.Y. Ismailov etc. // Multidisciplinary on-line scientific magazine of the Kuban State Agrarian University. - 2013. - Issue 85. - P. 43-56.

8. Patent no. 2099947 Russia, A01N63/00, C12N1/20, C12N1/20, C12R1:125. Biopreparation Phytosporin for protection of crops against diseases / Smirnov V.V., Sorokulova I.B., Berezhnitskaya T.G., Vanianz G.M., Menlikiyev M.Y., Nedorezkov V.D., Mineyev M.I., Vakhitov V.A., Baiguzina F. A. - no.96121980/13; Claimed on 15.11.1996; Published on 27.12.1997.

9. Patent no.2538157 Russia, C12N 1/20 A01N 63/00 C12R 1/125. Strain of bacteria Bacillus subtilis B 93 VIZR for protection of potato against diseases during storage / Novikova I.I., Boikova I.V., Pavlyushin V.A., Zeyruk V.N. - no.2013152716/10; Claimed on 27.11.2013; Published on 10.01.2015.

10. Patent no.2552146 Russia, C12N 1/20 A01N 63/02 C12R 1/125. Strain of bacteria Bacillus subtilis BZR 517 to make biopreparation against plant pathogenic fungi / Asaturova A.M., Dubyaga V.M. - no. 2013151375/10; Claimed on 20.11.2013; Published on 10.06.2015.

11. Patent no.2648163 Russia, C12N 1/20. Strain Bacillus mojavensis Lhv-97 possessing fungicidal and bactericidal activity / Dunaitsev I.A., Lev I.O., Klykova M.V., Zhigletsova S.K., Sosna I.M., Torgonina I.V., Varlamova T.A., Zaytseva S.D. - no. 2017128916; Claimed on 15.08.2017; Published on 22.03.2018.

12. Chebotar V.K., Zaplatkin A.N., Scherbakov A.V., etc. Bacterial preparations on basis of endophytic bacteria and rhizobacteria, which are promising for growth of productivity and efficiency of use of mineral fertilizers for summer barley (Hordeum vulgare L.) and vegetable crops // Selskohozyaystvennaya Biologiya. - 2016. - V. 51 — Issue 3. - P. 335-342.

1. Bacterial strain Bacillus mojavensis PS17 which possesses fungicidal activity with respect to plant pathogenic fungi and increases yield of agricultural crops, permits to broaden the spectrum of means of biological protection of plants against diseases, and was deposited in the National Bioresource Center - Russian National Collection of Industrial Microorganisms (NBC VKPM), NRC “Kurchatov Institute” - GosNIIgenetika under registration number VKPM B-13415.
